(19) United States
(12) Patent Application Publication
(10) Pub. No.: US 2010/0098792 A1
(43) Pub. Date: Apr. 22, 2010

(54) METHODS AND COMPOSITIONS USING IRVINGIA GABONENSIS TO REDUCE WEIGHT, CONTROL OBESITY, EFFECT ADIPOGENESIS, LOWER PPAR-GAMMA, LEPTIN, G3PDH AND TRIGLYCERIDES LEVELS AND INCREASE ADIPONECTIN LEVELS

(75) Inventor: Julius E. Oben, Yaounde (CM)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: GATEWAY HEALTH ALLIANCES, INC., Fairfield, CA (US)

(21) Appl. No.: 12/576,070

(22) Filed: Oct. 8, 2009

Related U.S. Application Data
(60) Provisional application No. 61/106,505, filed on Oct. 17, 2008.
(60) Provisional application No. 61/106,508, filed on Oct. 17, 2008.

(51) Int. Cl. A61K 36/00

(52) U.S. Cl. 424/776

(57) ABSTRACT

In one embodiment, a method is provided for lowering PPAR-Gamma in a mammal. The method comprises administering a composition containing an effective amount of Irvingia gabonensis seed to a mammal to reduce PPAR-Gamma levels in the mammal. In other embodiments, among other things, methods are provided for reducing triglyceride levels, inhibiting or reducing G3PDH activity, reducing body weight and increasing adiponectin levels in a mammal by administering Irvingia gabonensis.

### RELATED APPLICATION

This application claims the benefit of and priority to U.S. Provisional Application Ser. No. 61/106,505, filed Oct. 17, 2008, the contents of which are incorporated by reference herein in its entirety.

This application is related to U.S. Provisional Application Ser. No. 61/106,508, filed Oct. 17, 2008, the non-provisional of which is being filed concurrently with the present application.

### BACKGROUND

Endeavors to manage obesity have been heavily reliant on controlling energy intake and expenditure equilibrium, but have failed to curtail the overweight and obesity epidemic. This dynamic equilibrium is more complex than originally postulated and is influenced by lifestyle, calorie and nutrient intake, reward cravings and satiation, energy metabolism, stress response capabilities, immune metabolism and genetics. Fat metabolism is an important indicator of how efficiently and to what extent these factors are competently integrating.

Methods

Using murine 3T3-L1 adipocytes as a model for adipose cell biology research, the effects of IGOB131 were investigated on PPAR-Gamma, adiponectin, and leptin. These adipocytes were harvested eight days after the initiation of differentiation and treated with 0-250 μM of IGOB131 for 12 and 24 hours at 37 degrees C. in a humidified 5% CO₂ incubator. The relative expression of PPAR-Gamma, adiponectin, and leptin in 3T3-L1 adipocytes was quantified densitometrically using the software LabWorks 4.5, and calculated according to the reference bands of β-actin. An equivalent and effective dosage for a mammal to take to accomplish the same or similar results as those discussed in the present disclosure is believed to be 0.5 mg to 50 mg daily. More specifically, in at least one embodiment of the present disclosure, the effective amount of Irvingia gabonensis is approximately 1 mg to 10 mg daily. Acceptable and effective dosages of Irvingia gabonensis for mammals, especially humans, are also well known in the art.

Results

The IGOB131 significantly inhibited adipogenesis in adipocytes. The effect appears to be mediated through the down-regulated expression of adipogenic transcription factors (PPAR-Gamma) [P<0.05] and adipocyte-specific proteins (leptin) [P<0.05], and by up-regulated expression of adiponectin [P<0.05].

Conclusions

IGOB131 may play an important multifaceted role in the control of adipogenesis and have further implications in in-vivo anti obesity effects by targeting the PPAR-Gamma gene, a known contributory factor to obesity in humans.

### DETAILED DESCRIPTION

Endeavors to manage obesity have been heavily reliant on controlling energy intake and expenditure equilibrium, but have failed to curtail the overweight and obesity epidemic. This dynamic equilibrium is more complex than originally postulated and is influenced by lifestyle, calorie and nutrient intake, reward cravings and satiation, energy metabolism, stress response capabilities, immune metabolism and genetics. Fat metabolism is an important indicator of how efficiently and to what extent these factors are competently integrating. Obesity is a condition in which adipocytes accumulate a large amount of fat and become enlarged. It is characterized at the cellular level by an increase in the number and size of adipocytes differentiated from fibroblastic preadipocytes in adipose tissue [1].

Experiments were performed to determine, among other things, whether an OB131 Irvingia gabonensis seed extract (IGOB131) would provide a more beneficial comprehensive approach influencing multiple mechanisms and specifically PPAR-Gamma, leptin and adiponectin gene expressions, important in anti-obesity strategies.

The adipocyte is the primary site for energy storage, which accumulates triglycerides due to factors that include nutritional excess (energy imbalance), nutrient deficiencies, excessive stress, and genetic predispositions among other causes. Shimomura et al. [2] indicated that adipocytes synthesize and secrete biologically active molecules called adipocytokines. During adipocyte differentiation, transcriptional factors such as peroxisome proliferator-activated receptor gamma (PPAR-Gamma) are involved in the sequential expression of adipocyte-specific proteins [3]. Adiponectin is an adipocytokine that has been shown to have antiatherogenic, anti-inflammatory, and antidiabetic roles [4]. It has been found to be an important modulator of insulin sensitivity [5]. Nakamura et al. [6] indicated that high circulating levels of adiponectin might be protective against the development of coronary artery disease. Adiponectin levels are inversely correlated to body fat percentage, indicating that adiponectin plays an important role in fatty acid catabolism. Yamauchi et al. [7] indicated that adiponectin has emerged most recently as an important adipocytokine with insulin-sensitizing effects and represents a novel treatment target for insulin resistance and type 2 diabetes. Leptin is a secreted protein hormone that affects the hypothalamus to inhibit food intake and stimulates thermogenesis [8]. The cytosolic enzyme Glycerol-3-Phosphate Dehydrogenase (G3PDH) appears to have an important role catalyzing the conversion of glycerol into triglyceride [9].

In the present disclosure, the effects of an extract of OB131 Irvingia gabonensis (IGOB131) on the inhibition of intracellular triglyceride and G3PDH activity in 3T3-L1 adipocytes. We also examined the effect of these compounds on protein expression of adipogenesis in 3T3-L1 adipocytes.

Cell Culture

A murine 3T3-L1 cell line was used due to its widespread acceptance as a cell model for adipose cell biology research over the course of several decades [10]. 3T3-L1 preadipocytes (BCRC 60159) were purchased from the Bioresource Collection and Research Center (BCRC, Food Industry Research and Development Institute, Hsinchu, Taiwan, ROC). 3T3-L1 preadipocytes were planted into 6-well plates and maintained in DMEM supplemented with 10% bovine calf serum at 37 degrees C. in a humidified 5% CO₂ incubator. Adipocytic differentiation was induced by the adipogenic agents (0.5 mM IBMX, 1 μM DEX, and 1 μM INS) that were added to the culture medium. Afterwards, the medium was changed to normal culture medium and was freshly replaced every 48 hours. The cells were harvested eight days after the initiation of differentiation.

Triglyceride Content

Cells were incubated with 250 μM of IGOB131 for 72 hours at 37 degrees C. in a humidified 5% CO₂ incubator. Cells were collected and lysed in lysis buffer (1% Triton X-100 in PBS). The total triglyceride content in cells was determined using a commercial triglyceride assay kit (DiaSys Diagnostic Systems GmbH, Holzheim, Germany). The protein concentration was determined by using a BioRad DC protein assay kit (Bio-Rad Laboratories, Hercules, Calif.). Inhibition (%) was expressed as percent decrease in triglyceride content against control (0%).

Glycerol-3-Phosphate Dehydrogenase Activity

3T3-L1 adipocytes were harvested eight days after the initiation of differentiation and were incubated with 250 μM of IGOB131 for 72 hours at 37 degrees C. in a humidified 5% CO₂ incubator. Cells were washed twice with ice-cold PBS on 3T3-L1 adipocytes, and lysed in 25 mM Tris/1 mM EDTA, pH 7.5 for the measurement of glycerol-3-phosphate dehydrogenase (G3PDH) specific activity. G3PDH activity was determined according to the procedure of Wise and Green [11]. Protein concentration was determined by the BioRad DC protein assay kit (Bio-Rad Laboratories, Hercules, Calif.) using bovine serum albumin as a standard. Enzyme activity was expressed as units of activity/mg protein. Inhibition (%) was expressed as percent decrease in G3PDH activity against control (0%).

Western Blot Assay

Cells were incubated with 0-250 μM of IGOB131 acids for 12 and 24 hours at 37 degrees C. in a humidified 5% CO₂ incubator. They were collected and lysed in ice-cold lysis buffer (20 mM tris-HCl (pH 7.4), 2 mM EDTA, 500 μM sodium orthovanadate, 1% Triton X-100, 0.1% SDS, 10 mM NaF, 10 μg/mL leupeptin and 1 mM PMSF). The protein concentration was estimated with the Bio-Rad DC protein assay (Bio-Rad Laboratories, Hercules, Calif.) using bovine serum albumin as a standard. Total protein (50-60 μg) was separated by sodium dodecyl sulfate-polyacrylamide gel electrophoresis (SDS-PAGE) using a 12% polyacrylamide gel. The proteins in the gel were transferred to a PVDF membrane. The membrane was blocked with 5% skim milk in PBST (0.05% v/v Tween-20 in PBS, pH 7.2) for 1 hour. Membranes were incubated with primary antibody at 4 degrees C. overnight and then with secondary antibody for 1 hours. Membranes were washed in PBST for 10 minutes three times between each step. The signal was detected using the Amersham ECL system (Amersham-Pharmacia Biotech, Arlington Heights, Ill.). The relative expression of PPAR-Gamma, adiponectin, and leptin in 3T3-L1 adipocytes was quantified densitometrically using the software LabWorks 4.5, and calculated according to the reference bands of β-actin.

Statistical Analysis

Values are expressed as mean_S.E. For multiple comparisons, a one-way analysis of variance (ANOVA) was used. When ANOVA showed significant differences, post-hoc analysis was performed with the Newman-Keuls multiple range test using SPSS.

Results

Effect of IGOB131 on the Inhibition of Intracellular Triglycerides and G3PDH Activity in 3T3-I1 Adipocytes

The effect of IGOB131 on percent intracellular triglyceride and G3PDH levels were evaluated as indicated in the method section and the results are presented in Table 1. The reported values are the means±SD of three samples. Cellular harvesting and incubation was accomplished with IGOB131 as previously described in the method section. IGOB131 resulted in a significant inhibition of intracellular triglycerides (p<0.05). The peak inhibition using 250 μM of IGOB131 for 72 hours at 37 degrees C. in 5% CO₂ incubator was 80.9±0.7. Moreover, there was a similar finding utilizing the same parameters for the intracellular G3PHD levels. IGOB131 resulted in a significant inhibition of intracellular G3PDH (p<0.05). The peak inhibition using 250 μM of IGOB131 for 72 hours at 37 degrees C. in 5% CO₂ incubator was 71.6±1.2 (see Table 1).

TABLE 1
Effect of IGOB131 on the inhibition of Intracellular
Triglycerides and G3PDH activity in 3T3-I1 adipocytes.

| | % Intracellular | % Intracellular |
|---|---|---|
| Compound | Triglycerides Inhibition* | G3PDH Inhibition* |
| IGOB131 | 80.9 ± 0.7 | 71.6 ± 1.2 |

*Inhibitions (%) are expressed as percentages of the inhibition of control at 0%. 3T3-L1 adipocytes were harvested eight days after the initiation of differentiation. The cells were incubated with 250 μM of IGOB131 for 72 hours at 37 degrees C. in 5% CO₂ incubator. The reported values are the means ± SD (n = 3).

Effect of IGOB131 on protein levels of PPAR-Gamma, adiponectin, and leptin in 3T3-L1 adipocytes.

PPAR-Gamma

Effect of IGOB131 on protein levels of PPAR-Gamma, adiponectin, and leptin in 3T3-L1 adipocytes. 3T3-L1 adipocytes were harvested eight days after the initiation of differentiation. Cells were treated with 0-250 μM of IGOB131 for 12 and 24 hours at 37 degrees C. in a humidified 5% CO₂ incubator. The present experiment indicated that IGOB131 treatment significantly (P<0.05) inhibited the expression of PPAR-Gamma protein levels (FIG. 1).

Leptin

Effect of IGOB131 on protein levels of leptin in 3T3-L1 adipocytes. 3T3-L1 adipocytes were harvested eight days after the initiation of differentiation. Cells were treated with 0-250 μM of IGOB131 for 12 and 24 hours at 37 degrees C. in a humidified 5% CO₂ incubator. IGOB131 reduced the demand for excessive leptin synthesis, reducing circulating serum leptin levels (P<0.05). (FIG. 2)

Adiponectin

Effect of IGOB131 on Protein Levels of Adiponectin in 3T3-L1 Adipocytes.

3T3-L1 adipocytes were harvested eight days after the initiation of differentiation. Cells were treated with 0-250 μM of IGOB131 for 12 and 24 hours at 37 degrees C. in a humidified 5% CO₂ incubator. IGOB131 up-regulated the expression of Adiponectin (P<0.05). (FIG. 3)

Discussion

Over the past few decades, obesity has become a global epidemic in developed and developing countries. It is characterized by an increased adipose tissue mass and is associated with high health risk [11]. The prevalence of obesity and obesity-related disorders has led to major research interests in the influence of adipose tissue mass [12]. The 3T3-L1 cell line is widely used as a model of adipocyte differentiation and adipose biology. Wang and Jones [13] indicated that the decreased adipocytic lipogenesis is one of the mechanisms of proposed antiobesity. The experiments discussed below determined, among other things, the effects of IGOB131 on inhibiting adipogenesis in 3T3-L1 adipocytes. It is believed that the inhibitory effect resulted from the repression of adipocyte-specific protein expressions.

The inhibition of adipogenesis and adipocyte differentiation with IGOB131 was determined. The effects of IGOB131 on the inhibition of intracellular triglyceride and G3PDH activity in 3T3-L1 adipocytes were also determined. Fasting induces conversion of glycerol into triglyceride through an induction of several hepatic enzymes such as G3PDH and glycerol kinase. Tomiyama et al. [14] indicated that the expression of G3PDH is induced several-fold upon conversion of preadipocytes to adipocytes, which is the predominant substrate for triglyceride synthesis in adipose tissue. The data indicated, among other things, that the exposure of 3T3-L1 adipocytes to IGOB131 resulted in lower levels of intracellular triglycerides and G3PDH than other compounds tested (Table 1).

Adipose tissue is now known to produce and secrete a PPAR-Gamma, which has roles in the early stage of adipocyte differentiation, because they are transcriptional factors for numerous genes [14, 15]. Some studies have addressed the important role that PPAR-Gamma plays in the regulation of insulin sensitivity and glucose homeostasis [16]. The present experiment indicated that IGOB131 treatment inhibited the expression of PPAR-Gamma protein levels (FIG. 1), which demonstrated that adipogenesis was inhibited by affecting the transcriptional factor cascade upstream of PPAR-Gamma expression. Leptin (product of ob gene), which is secreted from adipocytes and gains access to the brain, reduces food intake, and increases energy expenditure [7]. Leptin that is unable to gain access to the brain due to CRP binding, resulting in leptin resistance, increases hypothalamic signaling for leptin synthesis, promoting higher levels of circulating serum leptin. Adiponectin is specifically expressed in white adipose tissues and is one of the most important adipocytokines. Adiponectin is an adipocytokine that has been shown to have antiatherogenic, anti-inflammatory and antidiabetic roles [5]. IGOB131 reduced the demand for excessive leptin synthesis, reducing circulating serum leptin levels, and stimulated the up-regulation of adiponectin at the protein level (FIGS. 2, 3). Adiponectin expression would, therefore, be regulated by PPAR-Gamma transcriptional activity [17].

Conclusions

The inhibitory effects of IGOB131 on 3T3-L1 adipocytes, as indicated by the decrease in intracellular triglyceride content and G3PDH activity, have been elucidated. It appears to be mediated through the down-regulated expression of adipogenic transcription factors (PPAR-Gamma) and adipocyte-specific proteins (leptin), and then the up-regulated expression of adiponectin. These results indicate that IGOB131 may play an important role in the control of adipogenesis and might have further implications in in-vivo antiobesity effects that exert specific influence on the PPAR-Gamma gene, a known contributory factor to obesity in humans [18]. This disclosure provides insight into an important mechanism for combating obesity.

### REFERENCES

1. Furuyashiki, T, Nagayasu H, Aoki Y, Bessho H, Hashimoto T, Kanazawa K, Ashida H: “Tea catechin suppresses adipocyte differentiation accompanied by down-regulation of PPAR-Gamma and C/EBPα in 3T3-L1 cells.” Biosci, Biotechnol Biochem 2004, 68:2353-2359.

2. Shimomura I, Funahashi T, Takahashi M, Maeda K, Kotani K, Nakamura T, Yamashita S, Miura M, Fukuda Y, Takemura K, Tokunaga K, Matsuzawa Y: Enhanced expression of PAI-1 in visceral fat: possible contributor to vascular disease in obesity. Nat Med 1996, 2:800-803.

3. Gregorie F M, Smas C M, Sul H S: Understanding adipocyte differentiation. Physiol Rev 1998, 78:783-809.

4. Pajvani U B, Du X, Combs T P, Berg A H, Rajala M W, Schulthess T, Engel J, Brownlee M, Scherer P E: Structure-function studies of the adipocyte-secreted hormone Acrp30/adiponectin. Implications for metabolic regulation and bioactivity. J Biol Chem 2003, 278:9073-9085.

5. Pajvani U B, Scherer P E: Adiponectin: systemic contributor to insulin sensitivity. Curr Diabetes Rep 2003, 3:207-213.

6. Nakamura Y, Shimada K, Fukuda D, Shimada Y, Ehara S, Hirose M, Kataoka T, Kamimori K, Shimodozono S, Kobayashi Y, Yoshiyama M, Takeuchi K, Yoshikawa J: Implications of plasma concentrations of adiponectin in patients with coronary artery disease. Heart 2004, 90:528-533.

7. Yamauchi T, Kamon J, Waki H, Terauchi Y, Kubota N, Hara K, Mori Y, Ide T, Murakami K, Tsuboyama-Kasaoka N, Ezaki O, Akanuma Y, Gavrilova O, Vinson C, Reitman M L, Kagechika H, Shudo K, Yoda M, Nakano Y, Tobe K, Nagai R, Kimura S, Tomita M, Froguel P, Kadowaki T: The fat-derived hormone adiponectin reverses insulin resistance associated with both lipoatrophy and obesity. Nat Med 2001, 7:941-946.

8. Ahima R S, Flier J S: Leptin. Annu Rev Physiol 2000, 62:413-437. Wise L S, Green H: Participation of one isozyme of cytosolic glycerophosphate dehydrogenase in the adipose conversion of 3T3 cells. J Biol Chem 1979, 254:273-275.

9. Green H, Kehinde O: Sublines of mouse 3T3 cells that accumulate lipid. Cell 1974, 1:113-116.

10. Wise, L. S.; Green, H. Participation of one isozyme of cytosolic glycerophosphate dehydrogenase in the adipose conversion of 3T3 cells. J. Biol. Chem 1979; 254: 273-275.

11. Seidell J C: Obesity, insulin resistance and diabetes—a worldwide epidemic. Br J Nutr 2001, 83:S5-S8.

12. Wang Y W, Jones P J: Conjugated linoleic acid and obesity control: efficacy and mechanisms. Int J Obes Relat Metab Disord 2004, 28:941-955.

13. Tomiyama K, Nakata H, Sasa H, Arimura S, Nishio E, Watanabe Y: Wortmannin, a specific phosphatidylinositol 3-kinase inhibitor, inhibits adipocytic differentiation of 3T3-L1 cells. Biochem Biophys Res Commun 1995, 212:263-269.

14. Rosen E D, Walkey C J, Puigserver P, Spiegelman B M: Transcriptional regulation of adipogenesis. Genes Dev 2000, 14:1293-1307.

15. Berger J, Moller D E: The mechanisms of action of PPARs. Annu Rev Med 2002, 53:409-435.

16. Gustafson B, Jack M M, Cushman S W, Smith U: Adiponectin gene activation by thiazolidinediones requires PPAR gamma 2, but not C/EBP alpha-evidence for differential regulation of the aP2 and adiponectin genes. Biochem Biophys Res Commun 2005, 308:933-939.

17. Blum K, Chen T J H, Meshkin B, Blum S H, Mengucci J F, Notaro A, Arcuri V, Waite R L, Braverman E R: The PPAR-Gamma Pro 12Ala allele polymorphism of the Peroxisome Proliferator Activated Receptor (γ) gene (PPARG2) is a risk factor with a self-identified obese Dutch population. Gene Ther Mol Biol 2007, 11:37-42.

### DRAWINGS

The above-mentioned features and objects of the present disclosure will become more apparent with reference to the following description taken in conjunction with the accompanying drawings wherein like reference numerals denote like elements and in which:

FIG. 1 shows the effect of IGOB131 on protein levels of PPAR-Gamma in 3T3-L1 adipocytes. 3T3-L1 adipocytes were harvested eight days after the initiation of differentiation. Cells were treated with 0-250 μM of IGOB131 for 12 and 24 hours at 37 degrees C. in a humidified 5% CO₂ incubator. The relative expression of PPAR-Gamma, in 3T3-L1 adipocytes was quantified densitometrically using the software LabWorks 4.5, and calculated according to the reference bands of β-actin. Values are means for three replicated cultures and *p<0.05 vs. control.

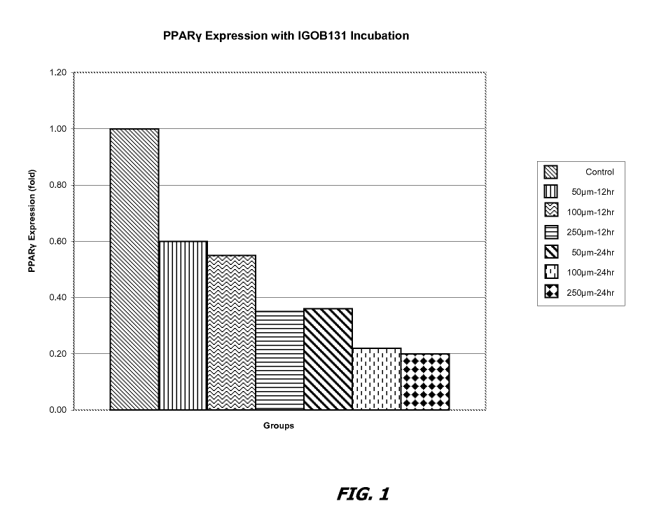

FIG. 2 shows the effect of IGOB131 on protein levels of Leptin in 3T3-L1 adipocytes. 3T3-L1 adipocytes were harvested eight days after the initiation of differentiation. Cells were treated with 0-250 μM of IGOB131 for 12 and 24 hours at 37 degrees C. in a humidified 5% CO₂ incubator. The relative expression of Leptin, in 3T3-L1 adipocytes was quantified densitometrically using the software LabWorks 4.5, and calculated according to the reference bands of β-actin. Values are means for three replicated cultures and *p<0.05 vs. control.

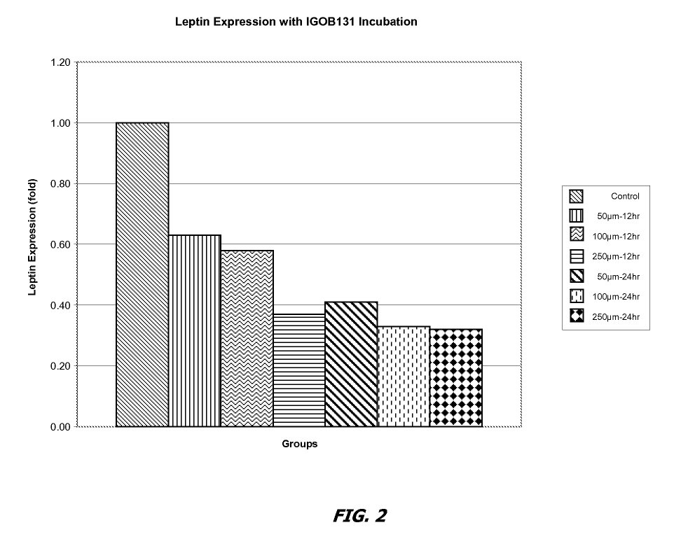

FIG. 3 shows the effect of IGOB131 on protein levels of Adiponectin in 3T3-L1 adipocytes. 3T3-L1 adipocytes were harvested eight days after the initiation of differentiation. Cells were treated with 0-250 μM of IGOB131 for 12 and 24 hours at 37 degrees C. in a humidified 5% CO₂ incubator. The relative expression of Adiponectin in 3T3-L1 adipocytes was quantified densitometrically using the software LabWorks 4.5 and calculated according to the reference bands of β-actin. Values are means for three replicated cultures and *p<0.05 vs. control.

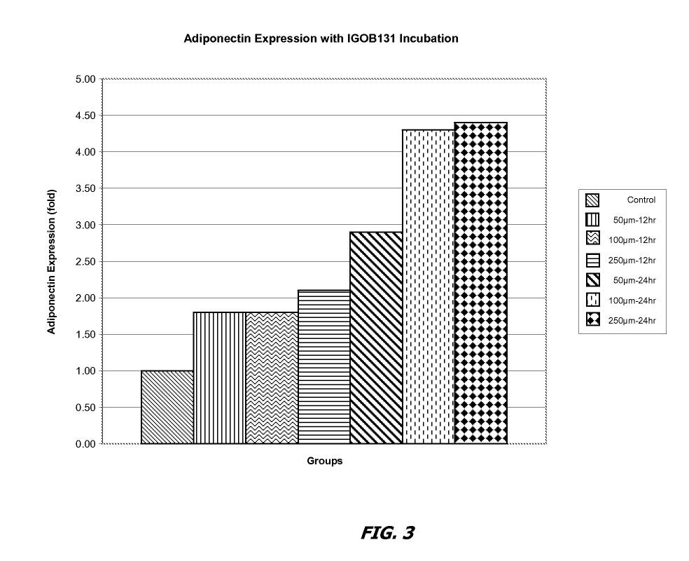

I claim:

1. A method for lowering PPAR-Gamma in a mammal, the method comprising:
administering a composition containing an effective amount of Irvingia gabonensis seed to a mammal to reduce PPAR-Gamma levels in the mammal.

2. A method of claim 1, wherein the effective amount of Irvingia gabonensis seed administered is approximately 0.5 mg to 50 mg daily.

3. A method of claim 1, wherein the effective amount of Irvingia gabonensis seed administered is approximately 1 mg to 10 mg daily.

4. A method of claim 1, wherein the effective amount of Irvingia gabonensis seed administered is approximately 50 mg to 1000 mg daily.

5. A method for reducing triglyceride levels in a mammal, the method comprising:
administering a composition containing an effective amount of Irvingia gabonensis seed to a mammal to reduce triglyceride levels in the mammal.

6. A method of claim 5, wherein the effective amount of Irvingia gabonensis seed administered is approximately 0.5 mg to 50 mg daily.

7. A method of claim 5, wherein the effective amount of Irvingia gabonensis seed administered is approximately 1 mg to 10 mg daily.

8. A method of claim 5, wherein the effective amount of Irvingia gabonensis seed administered is approximately 50 mg to 1000 mg daily.

9. A method for inhibiting or reducing G3PDH activity in a mammal, the method comprising:
administering a composition containing an effective amount of Irvingia gabonensis seed to a mammal to inhibiting or reducing G3PDH activity in the mammal.

10. A method of claim 9, wherein the effective amount of Irvingia gabonensis seed administered is approximately 0.5 mg to 50 mg daily.

11. A method of claim 9, wherein the effective amount of Irvingia gabonensis seed administered is approximately 1 mg to 10 mg daily.

12. A method of claim 9, wherein the effective amount of Irvingia gabonensis seed administered is approximately 50 mg to 1000 mg daily.

13. A method of claim 9, wherein the G3PDH activity is inhibited or reduced in adipocytes of the mammal.

14. A method for reducing body weight in a mammal, the method comprising:
administering a composition containing an effective amount of a purified and isolated Irvingia gabonensis seed extract to a mammal to reduce weight in the mammal.

15. A method of claim 14, wherein the effective amount of isolated and purified Irvingia gabonensis seed extract administered is approximately 0.5 mg to 50 mg daily.

16. A method of claim 14, wherein the effective amount of isolated and purified Irvingia gabonensis seed extract administered is approximately 1 mg to 10 mg daily.

17. A method of claim 14, wherein the effective amount of isolated and purified Irvingia gabonensis seed extract administered is approximately 50 mg to 1000 mg daily.

18. A method for increasing adiponectin levels in a mammal, the method comprising:
administering a composition containing an effective amount of Irvingia gabonensis seed extract to a mammal to increase adiponectin levels in the mammal.

19. A method of claim 18, wherein the effective amount of Irvingia gabonensis seed extract administered is approximately 0.5 mg to 50 mg daily.

20. A method of claim 18, wherein the effective amount of Irvingia gabonensis seed administered is approximately 50 mg to 1000 mg daily.
